(19) United States
(12) Patent Application Publication
(10) Pub. No.: US 2019/0142460 A1
(43) Pub. Date: May 16, 2019

(54) PLEATED TROCAR SEAL MEMBRANE

(71) Applicant: 5RMED TECHNOLOGY(CHENGDU) CO., LTD., CHENGDU (CN)

(72) Inventor: Moshu ZHU, CHENGDU (CN)

(73) Assignee: 5RMED TECHNOLOGY(CHENGDU) CO., LTD.

(21) Appl. No.: 16/249,898

(22) Filed: Jan. 17, 2019

Related U.S. Application Data
(63) Continuation of application No. PCT/CN2017/093601, filed on Jul. 20, 2017.

(30) Foreign Application Priority Data
Aug. 2, 2016 (CN) 201610622195.2

(51) Int. Cl. A61B 17/34

(52) U.S. Cl. CPC A61B 17/3439; A61B 2017/347; A61B 17/3498

(57) ABSTRACT

The invention discloses an improved pleated trocar seal membrane. Said seal membrane comprises a proximal opening, a distal aperture, and a sealing wall which extends from the distal aperture to the proximal opening, said distal aperture formed by a sealing lip for accommodating the inserted instrument and forming a gas-tight seal. Said sealing lip comprises a longitudinal axis and a transverse plane substantially perpendicular to said axis. Said sealing wall comprises a plurality of pleats extending laterally from the sealing lip. Each said pleat comprises a pleat peak, a pleat valley and a pleat wall extending there between. And the lip-adjacent area, the depth of the pleat wall gradually increases along the longitudinal axis; while outside the lip-adjacent area, the depth of which gradually decreases along the longitudinal axis. Said pleats enlarge hoop circumference, and reduce the cylinder hoop strain (stress) when a large diameter instrument is inserted, thereby reducing the hoop force and the frictional resistance.

### CROSS-REFERENCE TO RELATED APPLICATIONS

This application is a continuation of International Patent Application No. PCT/CN2017/093601 with a filing date of Jul. 20, 2017, designating the United States, now pending, and further claims priority to Chinese Patent Application No. 201610622195.2 with a filing date of Aug. 2, 2016. The content of the aforementioned applications, including any intervening amendments thereto, are incorporated herein by reference.

### TECHNICAL FIELD

The present invention relates to a minimally invasive surgical instrument, and in particular, to a trocar sealing element.

### BACKGROUND OF THE PRESENT INVENTION

A trocar is a surgical instrument, that is used to establish an artificial access in minimally invasive surgery (especially in rigid endoscopy). Trocars comprise in general a cannula and an obturator. The surgical use of trocars generally known as: first, make the initial skin incision at the trocar insertion site, then insert the obturator into the cannula, and then together they facilitated penetration of the abdominal wall through incision into the body cavity. Once penetrated into the body cavity, the obturator is removed, and the cannula will be left as access for the instrument get in/out of the body cavity.

In rigid endoscopy surgery, it is usually necessary to establish and maintain a stable pneumoperitoneum for the sufficient surgical operation space. The cannula comprises a sleeve, an outer body, a seal membrane (also known as instrument seal) and a duck bill (also known as closure valve). Said cannula providing a channel for the instrumentation in/out of the body cavity, said outer body connecting the sleeve, the duck bill and the seal membrane into a sealing system; said duck bill normally not providing sealing for the inserted instrument, but automatically closing and forming a seal when the instrument is removed; said seal membrane accomplishing a gas-tight seal against the instrument when it is inserted.

In a typical endoscopic procedure, it is usually set up 4 trocars (access), i.e. 2 sets of small diameter cannula (normally 5 mm in diameter), and 2 sets of large diameter cannula (normally 10˜12 mm in diameter). Instruments, in general passing through a small cannula are only for ancillary works; herein one large cannula as an endoscope channel, and the other large cannula as the main channel for surgeon to perform surgical procedures. Through said main channel thereof, 5 mm diameter instruments used in approximately 80% of the procedure, and said large cannula used in approximately 20% of the procedure; furthermore, 5 mm instruments and large diameter instruments need to be switched frequently. The small instruments are mostly used, so that the sealing reliability of which is more important. The large instruments are more preferably used in a critical stage of surgery (such as vascular closure and tissue suturing), therein switching convenience and operational comfort are more important.

FIG. 1 and FIG. 2 depict a typical 12 mm diameter cannula 100. Said cannula 100 comprises a lower housing 110, an upper housing 120, a seal membrane 130 which sandwiched between the lower housing 110 and the upper housing 120, and a duckbill seal 150. Said lower housing 110 including center hole 113 defined by an elongated tube 111. Said upper housing 120 including the proximal hole 123 defined by the inner wall 121. Said membrane 130 including a proximal opening 132, a distal aperture 133, a sealing lip 134, a frustum sealing wall 135, a flange 136 and an outer floating portion 137. Said distal opening 133 formed by a sealing lip 134. Said sealing lip 134 defining a longitudinal axis 141, transverse plane 142 substantially perpendicular to said axis 141; define the angle between the rotary-generating line (or generatrix) of the frustum sealing wall 135 and the transverse plane 142 as a guide angle ANG1.

As illustrated in FIG. 1, when a 5 mm diameter instrument inserted, it is approximately considered that only hoop force generated by the deformation of the sealing lip 34, ensures a reliable seal for the instrument. It is nevertheless favorable to operate the instrument from various extreme angles in surgery. There's a lot space left for the 5 mm-instrument to move radially in the 12 mm diameter cannula, so that greater radial force would be taken by the sealing lip 734. Therefore, the sealing lip 734 should have sufficient hoop force for the inserted 5 mm diameter instrument to ensure its sealing reliability thereof.

As illustrated in FIG. 2, drawing a cylinder of Di (Di>5 mm) to cut the sealing wall 135 forms an intersecting line 138. It is easy to understand for those skilled in the art, when an Di diameter instrument is inserted, the strain (stress) of said sealing wall 135 in the area from the sealing lip 134 to the intersecting line 138 will be larger, so the area refer to as lip-adjacent area (or stress concentration area). While the strain (stress) of said sealing wall 135 from the intersecting line 138 to the flange 136 is small. However, the different diameter (Di value) makes the boundary range of the lip-adjacent area (or stress concentration area) change larger or smaller. For the convenience of quantification, it is defined when Di is designed as the maximum diameter of the surgical instrument passing through the seal membrane, the area from the sealing lip 134 to the intersection line 138 is the lip-adjacent area.

As illustrated in FIG. 3, when a large diameter instrument is inserted (e.g. 12.8 mm), the sealing lip 134 will expand to a suitable size to accommodate the inserted instrument; said sealing wall 135 is divided into two portions: a conical wall 135c and a cylindrical wall 135d; said cylindrical wall 135d wrapped around the outer surface of the instrument to form a wrapped area with a high concentration of stress. Defining the intersecting line of the conical wall 135c and the cylindrical wall 135d as intersecting line 138a. When the instrument is removed, said sealing wall 135 return to natural state, and said intersecting line 138a spring-back to a ring radius of Dx, defined as intersecting line 138b, (not shown in FIG.); said intersecting line 138b is a bending boundary line when inserting a large diameter instrument. The angle between the rotary generating line of said conical wall 135c and the transverse plane 142 defines as ANG2, ANG2>ANG1; that is, when the large-diameter instrument is inserted, said sealing wall 135 rotates and stretches around its intersection line of said flange 136. Defining the height of the cylindrical wall 135d as Ha, not a fixed value; the factors such as different size of said distal aperture, different size of said sealing lip, different thickness of said sealing wall, different said guide angle or different diameter of inserted instrument, make Ha different.

The instrument inserted into the sealing membrane and moved during surgical procedure, there is large frictional resistance between the wrapped area and the inserted instrument. Said large frictional resistance is normally easy to cause the seal inversion, poor comfort of performance, fatigue performance, even result in cannula insecurely fixed on the patient's abdominal wall etc., such that the performance of cannula assembly is affected.

Among the defects caused by the large frictional resistance, the seal inversion is one of the most serious problems that affecting the performance of the cannula. As illustrated in FIG. 4, when a large diameter instrument is removed, easily cause seal inversion. When inversion happened, said sealing wall 135 divided into a cylindrical wall 135e, a conical wall 135f, and a conical wall 135g; said cylindrical wall 135e wrapped around the outer surface of the instrument to form a wrapped area with a high concentration of stress. Defining the height of the cylindrical wall 135e to be Hb, normally Hb>Ha; that is, the frictional resistance when the instrument is removed greater than it when the instrument is inserted, this difference affects the surgeon's operating feeling and even make the surgeon confused. More seriously, the inversion of the seal membrane may stretch into the proximal hole 123, that is the seal membrane positioned between the instrument and the inner wall 121 gets completely jammed. Measures for preventing the seal inversion are respectively disclosed in U.S. Pat. Nos. 7,112,185 and 7,591,802, and those measures can effectively reduce the probability of inversion but not completely solve the problem.

There are many factors affecting the frictional resistance, and the comprehensive effects of various factors must be considered in the perspective of mechanics and tribology. The seal membrane is preferably produced from rubber such as natural rubber, silicone or polyisoprene, its mechanical properties including super elastic and viscoelastic. Although the mechanical model of the rubber deformation process is complicated, it can still apply the generalized Hooke's law to describe approximately its elastic behavior, and Newton's internal friction law to describe the viscous behavior. Research suggests that the main factors affecting the friction of the two surfaces in contact between the rubber and the instrument include: the smaller the friction coefficient of said two surfaces, the smaller the friction is; the better lubrication condition of said two surfaces in contact, the friction smaller is; the smaller normal pressure of said two surfaces, the friction smaller is. Comprehensively considering the above factors, the present invention proposes better solutions for reducing the frictional resistance between the seal membrane and the inserted instrument.

In addition to said frictional resistance greatly affecting the performance of the cannula assembly, the stick-slip of the seal membrane is another main factor affecting the performance of trocar. Said stick-slip means that when the instrument moves longitudinally in the sleeve, the sealing lip and lip-adjacent area sometimes are relatively statically attached to the instrument (at this point, the friction between the instrument and the seal membrane is mainly static friction.); but sometimes it produced a relatively slippery phenomenon with the instrument (at this point, the friction between the instrument and the seal membrane is mainly dynamic friction.); and said static friction is much greater than said dynamic friction. The two frictions alternately occur, which causes the movement resistance and speed of the instrument in the seal membrane to be unstable. It is easy to be understood for those skilled in the art, that in minimally invasive surgery the surgeon can only use surgical instruments to touch (feel) the patient's organs and observe a part of the working head of the instruments through endoscopic image system. In this case where the vision is limited and it cannot be touched, the surgeon typically uses the feedback of the resistance when moving instruments as one of the information to judge whether the operation is abnormal nor not. The stick-slip affects the comfort of operation, the accuracy of positioning, and even induces the surgeon to make false judgment.

During the surgical application of the cannula, the stick-slip is difficult to avoid, but can be reduced. Researches have shown that said stick-slip is affected by two main factors: one is that the smaller the difference between the maximum static friction and the dynamic friction, the weaker the stick-slip is; the other is that the larger the axial tensile stiffness of the seal membrane, the weaker the stick-slip is. Avoiding excessive the hoop force between the seal membrane and the instrument, reducing the two surfaces contacted, maintaining good lubrication, respectively, can reduce the difference between the maximum static friction and the dynamic friction, thereby reducing stick-slip, meanwhile, increasing the axial tensile stiffness of the seal membrane also helps to reduce the stick-slip phenomenon. The invention also proposes measures for improving stick-slip.

A pleated seal membrane is disclosed in U.S. Pat. No. 7,789,861 (Chinses Patent Family CN101478924B). As illustrated in FIG. 5-10, said seal membrane 80 comprises an opening 81 defined by a lip 82. A plurality of pleats 89 are circumscribed with said opening 81 and extend laterally from said opening 81. Said pleats 89 are conically arranged. A wall section 85 circumscribes and is connected to the pleats 89. Each pleat 89 includes a pleat wall which extends between the pleat peak 84 and the pleat valley 83. The height of the pleat walls can be measured along the wall surface from the peak 84 to the valley 83. Said pleat walls increase in height as the pleats extend laterally from the opening 81. The lip 82 has a cylindrical portion, which when intersected with the pleats 89 results in a line 87, which defines a triangular region 89a pointing distally to the tip, corresponding to each peak 84. Said wall section 85 intersects the pleats 89 and form an intersection line 88; said line 88 defines a triangular region 85a pointing distally to the tip, corresponding to each valley 83. The advantages thereof, lie in that said pleats help to reduce hoop stresses when an instrument is positioned in the opening 81, thereby reducing friction between the instrument and the seal membrane. Reducing hoop forces facilitates a thicker wall thickness to be used, while providing similar or reduced tensile force than non-pleated lip seal designs.

The geometry of the pleats (89) can be designed to minimize or eliminate hoop stress in the pleated portion of the seal membrane 80 when an instrument is introduced. This geometric relationship, herein conforms to the following equation:

$$h \geq \frac{π}{P} \sqrt{{r}^{2} + {r}_{i}^{2} - {r}_{id}^{2}}$$

### Where:

h=pleat wall height as a function of radius
r=radius
ri=the largest radius designed for the insertion through the seal membrane
rid=radius at inside diameter of pleat section of the seal membrane
P=number of pleats

In the embodiment disclosed in U.S. Pat. No. 7,798,671, the inner diameter of the opening 81 in the relaxed state is between 3.8˜4.0 mm. The elasticity of the seal membrane 80 is sufficient to ensure that the opening 81 can be expanded to gas-tightly engage a surgical instrument with 12.9 mm diameter. The seal membrane 80 contains 8 linear pleats 89. Therefore, h in this embodiment should conform to the following formula:

h≥3.14/8\sqrt{r²+(6.45)²−2²}

Theoretically increasing the number of pleats 89 can reduce said h. In the prior art described above, the non-pleated seal membrane is usually designed to have the thickness of the wall 0.5 to 0.7 mm. If a pleated seal membrane is used to reduce the hoop force, it is advantageous to use thicker pleat walls. That is, if the thickness of the seal membrane is greater than 0.5, the number of pleats is usually not more than 8, otherwise it cannot be manufactured. The circumference of the opening 81 is usually 11.9˜12.5 mm diameter, and the thickness of each pleat wall is usually not less than 0.5 mm. 8 pleats have 16 pleat walls in total, and more pleats will make manufacturing very difficult or impossible to manufacture. Therefore, the manufacturable seal membrane conforming to this formula has h≥2.4 mm.

The schematic diagram of the seal membrane in the U.S. Pat. No. 7,789,861 does not conform to the above formula. As illustrated in FIG. 5-9 said pleats 80 the above formula, h at the opening 81 is equal to 2.4 mm diameter (i.e., the length of the intersection line 87 is equal to 2.4 mm diameter).

Refer to FIGS. 8 and 9, along the outer annular wall of the lip 82 making a cylindrical split surface S1 (not shown) to divide said seal membrane 80 into two parts, a lip section 82a and a seal membrane section 80a. The split surface S1 cuts the pleats 89 to form intersection lines 87a, 87b; The length of said intersection line 87a is approximately equal to the length h of said intersection line 87 (h=2.4 mm). It is not difficult to understand with reference to FIG. 8 and the previous formula, that when said h≥2.4 mm diameter, if the 12.9 mm diameter instrument is inserted, the change in the shape of the pleats 89 of the seal membrane 80a mainly is shown as local bending deformation and macroscopic displacement, rather than the overall microscopic molecular chain elongation and overall tensile deformation.

Refer to FIG. 9, when the h≥2.4 mm diameter, the lip 82a opposite to the lip 82 increases several triangle areas 89a. When a 5 mm diameter instrument is inserted, mainly relying on the hoop tightening force generated by the circumferential deformation of the lip 82 to ensure the sealing reliability, said triangular region 89a is generally not sealed to the inserted 5 mm diameter instrument. However, when the 12.9 mm diameter instrument is inserted, the triangular region 89a generates a large tensile deformation and is partially wrapped on the outer surface of the instrument, increasing the actual contact area of the two surfaces between the instrument and the seal membrane. It is easy to understand for those skilled in the art that although the analysis of the lip section 82a and the seal membrane 80a separated proves that the larger h is, the smaller hoop stresses of said pleats 89 is, when which is considered as a whole, this is not the case. Inappropriate height will increase the actual contact area of the two surfaces between the seal membrane and the instrument, thereby increasing the frictional resistance.

Refer to FIG. 6-8, It is easy to understand for those skilled in the art that if the geometric size of the pleats meets the above formula (h≥2.4 mm), that is, from near the lip, the hoop circumference of the pleats is already larger than the peripheral circumference of the inserted instrument, so it is not necessary to adopt increasing pleats. Moreover, in this case of increasing pleats used, that is, the shape of each pleat wall is approximately trapezoidal (refer to FIG. 6-7). When the large diameter instrument is inserted to allow the pleat wall to be diastolic, said the pleat wall will be bended and rotary around the intersection of the pleats 89 and the wall section 85, and the bending and rotation caused by trapezoidal pleat wall in the sealing lip and the lip-adjacent area are inconsistent relative to the bending arm or the rotary arm of the lip; thereby increasing the additional deformation force, at the same time, causing axial elongation instability in the lip and its adjacent area (different insertion angles of the instrument, different axial elongation), and causing the aforementioned stick-slip phenomenon more significant.

FIG. 10 depicts a pleated seal membrane 80b that does not conform to the aforementioned equation. Said seal membrane 80b has a pleat that gradually increases in the axial direction from the lip; while the geometric size of the pleats of the seal membrane 80b is small and does not conform to the aforementioned formula. Said seal membrane 80b and said the seal membrane 80 have similar geometries, which differ only in the geometric size. It is easy to understand for those skilled in the art that, if you do not limit the geometric size, smaller pleats that extend laterally from the lip and gradually increase can not play a significant role in reducing the hoop force.

In summary, the pleated trocar seal disclosed in U.S. Pat. No. 7,789,861 is not incomplete. Further analyzing the complexity of the clinical application of the trocar, and comprehensively considering effects of various factors, the invention proposes improved pleated trocar seal.

### SUMMARY OF PRESENT INVENTION

In conclusion, one object of the invention is to provide a trocar seal membrane, said seal membrane comprises a proximal opening, a distal aperture, a sealing lip, and a sealing wall from the distal aperture extending to the proximal opening, said distal aperture formed by a sealing lip for accommodating the inserted instrument and forming a gas-tight seal. Said the sealing wall includes a proximal surface and a distal surface. Said seal membrane can ensure a reliable seal for the inserted 5 mm instrument, and reduce frictional resistance and improve stick-slip when a large-diameter instrument is inserted.

As described in the background, the wrapped area formed by the sealing lip and the lip-adjacent area when a large diameter instrument inserted, is the major factor cause of frictional resistance. For reducing said frictional resistance, comprehensive consideration should be given such as reducing the radial stress between the instrument and the seal membrane, reducing said wrapped area, and reducing the actual contact area of the two surfaces. It is easy to understand for those skilled in the art that in accordance with the generalized Hooke's law and Poisson effect, enlarge hoop circumference, and reduce hoop strain (stress), thereby reducing radial strain (stress). But it should be noted that it is impossible to enlarging the hoop circumference in order to reduce the strain of the sealing lip which will result in reduced sealing reliability when applying 5 mm instruments. Since the stress in the lip-adjacent area is highly concentrated when applying a large diameter instrument, the hoop circumference of the lip-adjacent area should be rapidly increased. In regard to outside the lip-adjacent area, since the strain (stress) is small, it is not necessary to adopt measures to enlarge the hoop circumference. In addition, enlarging the hoop circumference, in the meantime increasing the axial tensile stiffness in the lip-adjacent area and maintain good lubrication (reducing difference between the maximum static friction and dynamic friction), thereby the stick slip in the lip-adjacent area is improved.

In one aspect of the present invention, said seal membrane comprises a proximal opening, a distal aperture, and a sealing wall from the distal aperture extending to the proximal opening, said distal aperture formed by a sealing lip for accommodating the inserted instrument and forming a gas-tight seal. Said sealing lip comprises a longitudinal axis and a transverse plane substantially perpendicular to said axis. Said sealing wall comprises a plurality of pleats extending laterally from the sealing lip. Each said pleat comprises a pleat peak, a pleat valley and a pleat wall extending there between. And in the lip-adjacent area, the depth of the pleat wall gradually increases along the longitudinal axis; while outside the lip-adjacent area, the depth of which gradually decreases along the longitudinal axis.

Alternatively, the angle between said pleat peak and said pleat valley relative to said transverse plane conforms to the following equation:

$$tan β - tan α \geq \sqrt{{( \frac{π {R}_{i}}{PR} )}^{2} + 2 ( cos ( 180 / P ) - 1 )}$$

### Where:

tan=tan function
cos=cosine function
P=number of pleats
R=The distance from the pleat as the starting point for measurement to the central axis of the sealing lip
Ri=the largest radius designed for the surgical instrument through the seal membrane
β=the angle between the pleat peak and the transverse plane
α=the angle between the pleat valley and the transverse plane

By theoretical analysis and related research, it is shown that reducing the value of the guiding angle α is advantageous for reducing the length of said wrapped area. In an optional embodiment, 8 pleats are adopted; the angle between said pleat valley and the transverse plane is 0°≤α≤25°. In another optional embodiment, thickened pleat peaks are adopted. Said thickened pleat peak, that is, the thickness of the wall at the pleat peak is greater than the thickness of the pleat wall. The thickened pleat peak acts as reinforcing ribs, a plurality of thickened pleat peaks together to strengthen the axial tensile stiffness of the sealing wall. Since the pleats enlarge the hoop circumference in the lip-adjacent area, the thickened pleat peaks enhance the axial tensile stiffness without significantly increasing the hoop tensile stiffness; that is, increasing the axial stiffness without increasing the hoop force, such that which can effectively reduce the stick-slip described in the background.

In another aspect of the present invention, said seal membrane comprises a proximal opening, a distal aperture, and a sealing wall from the distal aperture extending to the proximal opening; said distal aperture formed by a sealing lip for accommodating the inserted instrument and forming a gas-tight seal; said sealing lip, which is cylindrical, comprises a longitudinal axis and a transverse plane substantially perpendicular to said axis. Said sealing wall comprises a plurality of pleats extending laterally from the sealing lip; each said pleat comprises a pleat peak, a pleat valley and a pleat wall extending there between. Said seal membrane comprises a flange and a conical sidewall extending from the flange; said conical sidewall and said pleat are intersected. When said pleats extending laterally outward, in the lip-adjacent area the depth of said pleats gradually increases along the longitudinal axis; outside the lip-adjacent area the depth of said pleats gradually decreases along the longitudinal axis. Said seal membrane also includes an outer floating portion extending from the flange to the proximal opening. Optionally, the thickness of the conical sidewall is less than the thickness of the pleat wall.

The other object of the invention is to provide a trocar seal assembly. Said seal includes a lower retainer ring, a seal membrane, a protector, an upper retainer ring, an upper body, an upper cover; said seal membrane and said protector device are sandwiched between the upper retainer ring and the lower retainer ring, said 4 mutually overlapping protectors used to protect the seal membrane from sharp edges of the inserted instrument. The proximal opening of said the seal membrane sandwiched between the upper body and the upper cover, said outer floating portion makes the seal membrane and protector float laterally in the housing formed by the upper body and the cover.

It is believed that the above invention or other objects, features and advantages will be understood with the drawings and detailed description.

### DESCRIPTION OF THE DRAWINGS

A more complete appreciation of this invention, and many of the attendant advantages thereof will be readily apparent as the same becomes better understood by reference to the following detailed description, where:

FIG. 1: shows a simulated distorted view of the cannula with the 5 mm diameter instrument inserted in the prior art;

FIG. 2: shows a detailed view of the seal membrane 730 in the prior art;

FIG. 3: shows a simulated distorted view of the cannula with the 12.8 mm diameter instrument inserted in the prior art;

FIG. 4: shows a simulated distorted view of the cannula with the 12.8 mm diameter instrument removed in the prior art;

FIG. 5: shows a 3D perspective view of the seal membrane 80 according to another prior art;

FIG. 6: shows a sectional view along line 6-6 in FIG. 5 of the prior art:

FIG. 7: shows a sectional view along line 7-7 in FIG. 5 of the prior art:

FIG. 8-9: shows a segmentation view of the seal membrane after the circumferential cutting separation in FIG. 5 of the prior art;

FIG. 10: shows a 3D perspective view of the seal membrane 80a according to another prior art.

FIG. 11: shows a 3D perspective partial sectional view of the cannula in the invention;

FIG. 12: shows an exploded view of the seal membrane assembly in the cannula in FIG. 11;

FIG. 13: shows a 3D perspective partial sectional view of the seal membrane assembly in FIG. 12;

FIG. 14: shows a 3D inside perspective view of the seal membrane 330 without the proximal end and floating portion in FIG. 12.

FIG. 15: shows a 3D outside perspective view of the seal membrane 330 without the proximal end and floating portion in FIG. 12.

FIG. 16: shows a sectional view along-line 16-16 in FIG. 14

FIG. 17: shows a sectional view along-line 17-17 in FIG. 14

FIG. 18-19: shows a segmentation view of the seal membrane after the circumferential cutting separation in FIG. 15;

FIG. 20: shows a simulated distorted view of the seal membrane with the 12.8 mm instrument inserted in FIG. 14;

FIG. 21: shows a view with the 12.8 mm inserted instrument hidden

FIG. 22: shows a 3D inside perspective view of the seal membrane 430 without the proximal end and floating portion in the second embodiment.

FIG. 23: shows a 3D outside perspective view of the seal membrane 430 without the proximal end and floating portion in the second embodiment.

FIG. 24: shows a sectional view along-line 24-24 in FIG. 22

FIG. 25: shows a sectional view along-line 25-25 in FIG. 22

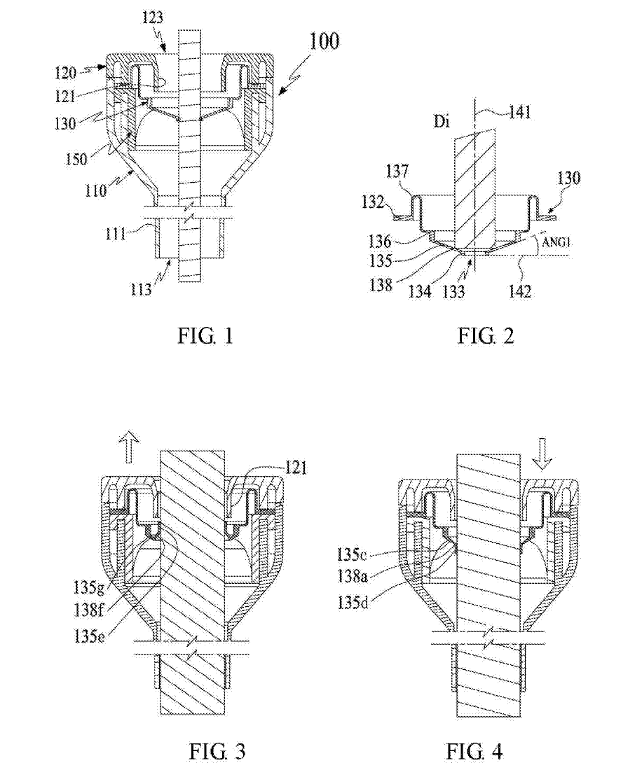

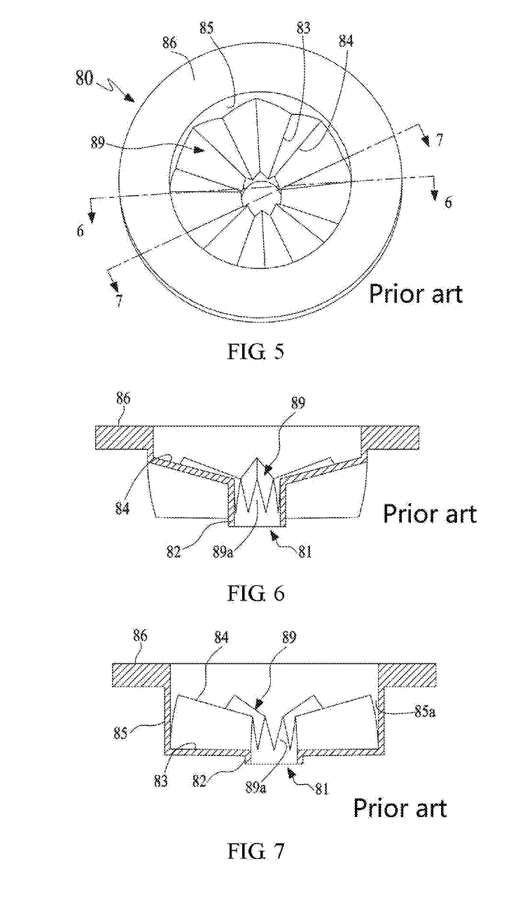

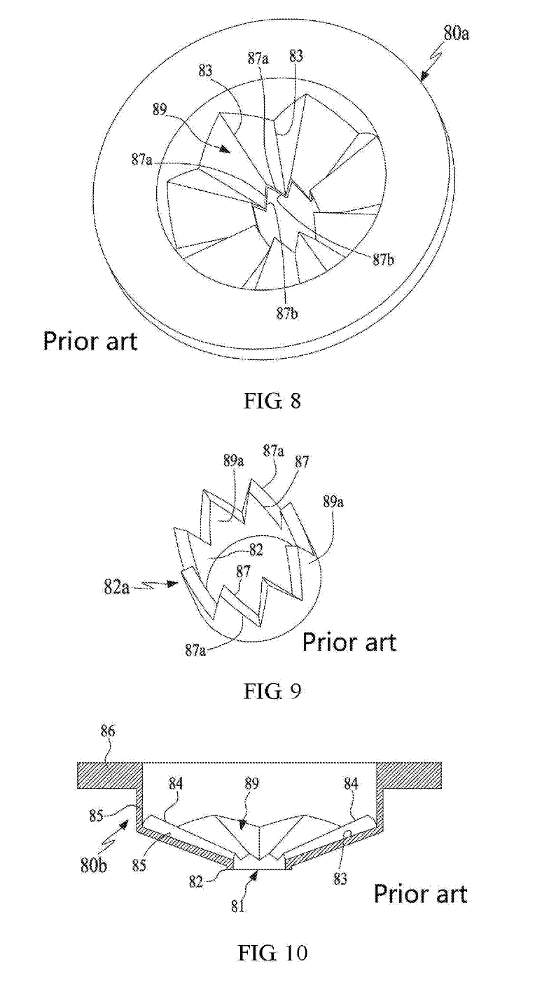

In all views, the same referred number shows the same element or assembly.

### DETAILED DESCRIPTION OF PREFERRED EMBODIMENTS

Embodiments of the invention are disclosed herein, however, it should be understood that the disclosed embodiments are merely examples of the invention, which may be implemented in different ways. Therefore, the invention is not intended to be limited to the detail shown, rather, it is only considered as the basis of the claims and the basis for teaching those skilled in the art how to use the invention.

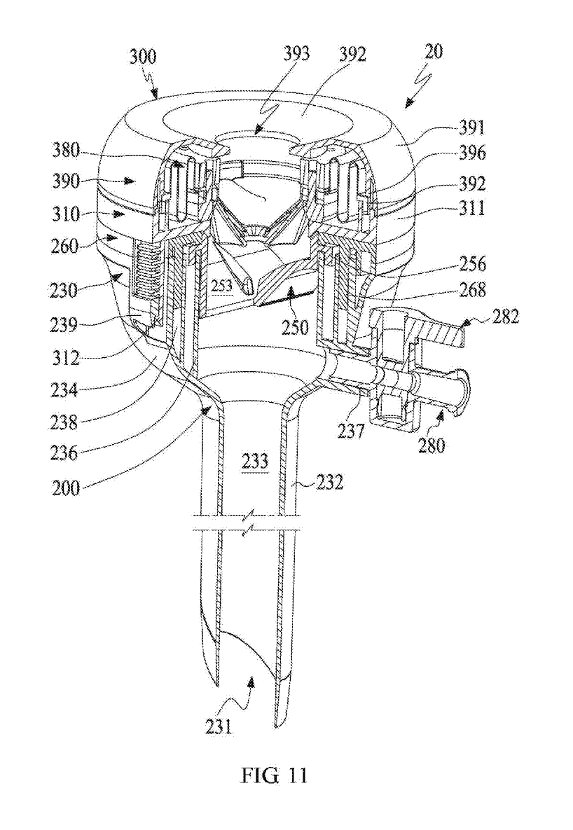

FIG. 11 shows an overall view of the structure of trocar. A typical trocar comprises an obturator 10 (not shown) and a cannula 20. The cannula 20 comprises an open proximal end 392 and an open distal end 231. In a typical embodiment, said obturator 10 passes through said cannula 20, together they facilitated penetration of the abdominal wall through incision into the body cavity. Once penetrated into the body cavity, the obturator 10 is removed, and the cannula 20 will be left as access for the instrument get in/out of the body cavity. Said proximal end 392 in the external position of the patient and said distal end 231 in the internal position of the patient. A preferred cannula 20 can be divided into the first seal assembly 200 and the second seal assembly 400. Locking receptacle 239 in said seal assembly 300 can be locked with snap-in projection 312 in said seal assembly 300. The cooperation of snap-in projection 312 and the locking receptacle 239 can be quick release by one hand. The main purpose is for convenience of taking out tissues or foreign matter from the patient in the surgery. There are multiple ways to implement the quick release connection of said seal assembly 200 and assembly 300. In addition to the structure shown in this embodiment, a threaded connection, a rotary snap-in or other quick lock structure also may be applied. Alternatively, said assembly 200 and assembly 300 can be designed as a structure that can not be split quickly.

FIG. 11 shows the composition and assembly relationship of the first seal assembly 200. The lower body 230 includes an elongated tube 232, which defines the sleeve 233 passed through the distal end 231 and is connected to the outer housing 234. Said lower body 230 comprises an inner wall 236 supporting duck bill seal and a valve bore 237 that communicates with the inner wall 236. The plunger 282 mounted in the valve body 280, the said two are mounted into said valve bore 237. The flange 256 of the duck bill seal 250 is sandwiched between the inner wall 236 and the lower cover 260. There are various ways of fixing between the lower cover 260 and the lower body 230, such as the interference fit, ultrasonic welding, glue bonding, and snap fastening. 4 cylinders 268 of said lower cover 260, in this embodiment, 4 holes 238 of said lower body 230 are adopted to interference fit, so that the duckbill seal 250 is in the compressed state. Said tube 232, said the inner wall 236, said duck bill seal 250, said valve body 280 and said plunger 282 together are comprised the first chamber. Said duck bill seal 250, in this embodiment, is a single-slit, while other types of closure valves may also be used, including flapper valves, multi-silted duck bill valves. When the instrument is passed through said duck bill seal 250, the duckbill 253 will be opened, but it generally does not provide a complete seal against the instrument. When the instrument is removed, said duckbill 253 closed and substantially prevents insufflation fluid from escaping through the first chamber.

FIG. 11 shows the composition and assembly relationship of the second seal assembly 300. The seal membrane assembly 380 is sandwiched between the upper cover 310 and the upper body 390. The proximal end 332 of the seal membrane assembly 380 is secured between the inner ring 316 of the upper cover 310 and the inner ring 396 of the upper body 390. There are various secured ways between the upper cover 390 and the upper body 310, such as the interference fit, ultrasonic welding, glue bonding, and snap fastening. The connection method, shown in this embodiment, is the outer shell 391 of the upper body 390 and the outer shell 311 of the upper cover 310 are secured by ultrasonic welding, so that the proximal end 332 of the seal membrane assembly 380 is in the compressed state. The center hole 313 of said upper cover 310, said inner ring 316, and said seal membrane assembly 380 together are comprised the second chamber

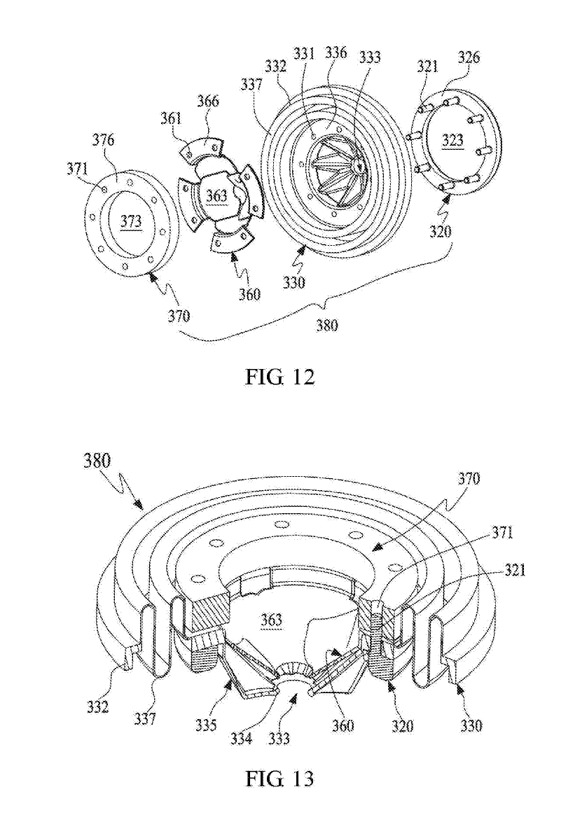

FIG. 12-13 illustrate the composition and assembly relationship of said seal membrane assembly 380, which including a lower retainer ring 320, a seal membrane 330, a protection device 360 and an upper retainer ring 370. Said the seal membrane 330 and said protection device 360 are sandwiched between the lower retainer ring 320 and the upper retainer ring 370, moreover, the cylinder 321 of the said lower retainer ring 320 is aligned with corresponding holes on other components in said seal membrane assembly 380. Said cylinder 321 and the bore 371 of the upper retainer ring 370 are adopted to interference fit, so that the whole seal membrane assembly 380 in the compressed state. Said protection device 360 includes 4 protectors 363 arranged so as to protect a center sealing body of said seal membrane 330, herein permit the sharp edge of the instrument to pass through without causing perforations or tears to the seal membrane 330.

Said seal membrane 330 includes a proximal opening 332, a distal end aperture 333, and the sealing wall extending from the distal end to the proximal end, said sealing wall including a proximal surface and a distal surface. Said aperture 333 formed by a sealing lip 334 for accommodating an inserted instrument and forming a gas-tight seal. Said sealing lip 334 may be non-circular. As described in the background of the invention, the circumference of the sealing lip should be short and strong enough to ensure sealing reliability when a 5 mm diameter instrument is inserted. In the present embodiment, the sealing lip 334 is circular, defining its radius as Rlip, so that the circumference of the sealing lip is approximately equal to 2*Rlip*π (π=3.14159), usually the circumference of the sealing lip is 11.8˜13.8 mm. The cross-section of said sealing lip is circular, usually its radius is 0.7 to 1.0 mm diameter.

Said the seal membrane 330 also including the flange 336; The sealing wall 335 has one end connected to the sealing lip 334 and the other end connected to the flange 336; a floating portion 337 has one end connected to the flange 336 and the other end connected to said proximal end 332. Said flange 336 can be applied for mounting the protector device 360. Said floating portion 337 including one or several plurality of radial (lateral) pleats such that the entire seal membrane assembly 380 can float in the assembly 300.

Said assembly 380 can be made from a variety of material s with a range of different properties. For instance, said seal membrane 330 is made of a super elastic material such as silicone or polyisoprene; said protector device 360 is made of a semi-rigid thermoplastic elastomer; and said lower retainer ring 320 and said upper retainer ring 370 are made of a relatively hard rigid material such as polycarbonate.

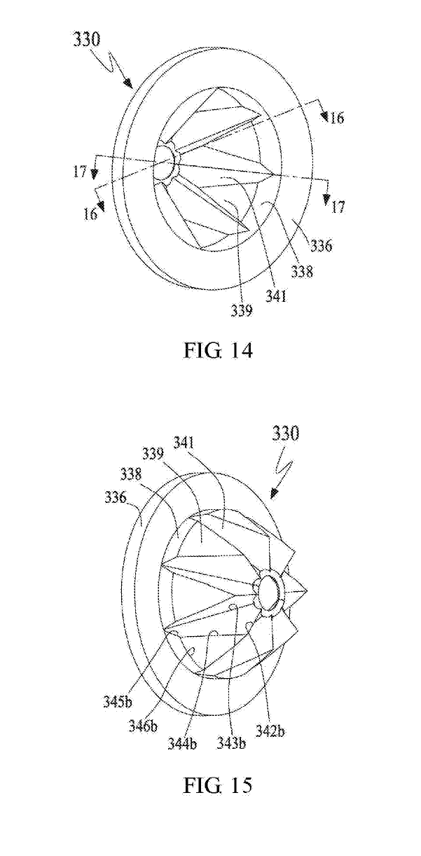

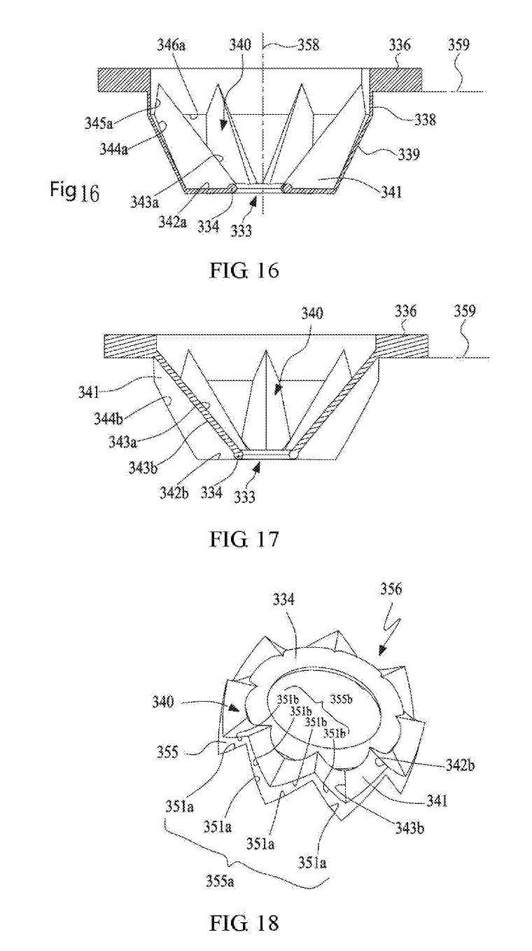

FIG. 14-17 show more detailed depiction the seal membrane 330 of the first embodiment of the invention. In order to reduce the production cost, the seal membrane 330 is preferably designed as a monolithic part, but can also be designed as an inner seal body and an outer floating portion, separated from the flange 336. The first embodiment is mainly directed to the improvement of the inner seal body. To simplify the description, the outer floating portion and the proximal end are not shown in the subsequent description of the seal membrane.

Said sealing lip 134 comprising a longitudinal axis 158, and a transverse plane 159 that is generally perpendicular to the longitudinal axis 158. Said sealing wall 335 includes a plurality of pleats 340. The pleats 340 and the sealing lip 334 are circumscribed and extend laterally away from the axis 358. Said pleats 340 include pleat valleys 342a, 342b; pleat peaks 343a, 343b; and a pleat wall 341. Sealing wall 335 includes 8 said linear pleats 340, in the present embodiment, although a more or less number of pleats can be employed. In the present embodiment, said pleats 340 are conically arranged around the sealing lip 334. Said pleats 340 intersect the flange 336 and its extended wall 338 to form an intersection line 345a, 345b. A part of the frustum wall 339 intersects the pleat wall 341 to form an intersection line 344a, 344b; The frustum wall 339 intersects the extended wall 338 to form an intersection line 346a, 346b. Defining the angle between the pleat valley 342a (342b) and the transverse plane surface 359 as a guide angle α; Defining the angle between the pleat peak 343a(343b) and the transverse plane surface 359 as a guide angle β; Defining the angle between the pleat valley 342a (342b) and the pleat peak 343a (343b) as a wave angle θ; and ranges of them are from 0° to 90°.

When the pleats 340 extending laterally outward, in the lip-adjacent area, the depth of said pleat wall 341 gradually increases along the longitudinal axis; outside the lip-adjacent area the depth of said pleats 341 gradually decreases along the longitudinal axis. The height of the pleat wall can be measured along the wall surface between the pleat valley 342a (342b) and the pleat peak 343a (343b).

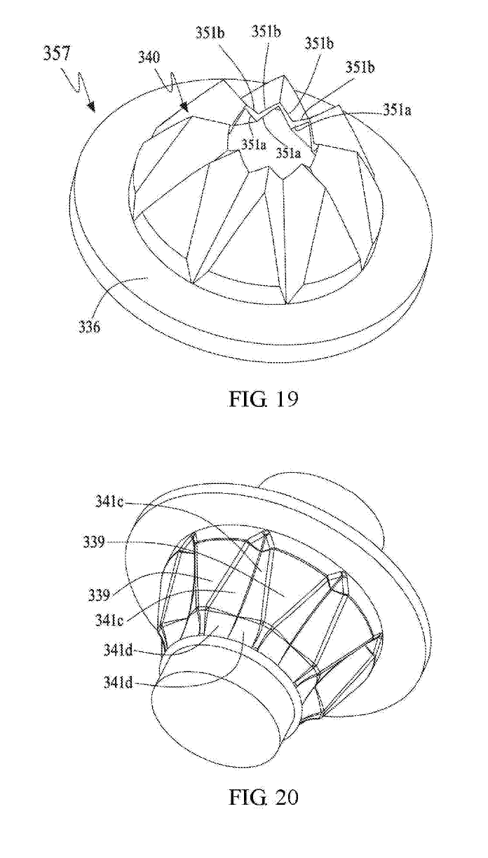

Taking the longitudinal axis 358 as a rotary axis, making a cylindrical surface (not shown) with a radius RI divides the seal membrane 330 into an inner portion 356 (as in FIG. 18) and an outer portion 357 (FIG. 19). Said cylindrical surface intersects said pleat wall 341 to form a plurality of intersection lines 351a and 351b. The plurality of segments 351a are formed an annular intersection line 155a; the plurality of segments 351b are formed an annular intersection line 155b, and the section 355 defined by said annular intersection line 355a and 355b.

As shown in FIG. 18-19, it is obvious that the circumference L1 of the intersection line 355a (355b) is much larger than 2*π*R1, that means the reverse concave-furrow plays a role in enlarging hoop circumference. Those skilled in the art can understand that there must be some R1 value making the outer portion 357, which is divided by the cutting plane M1, to start from the section 355, the main change of its shape is shown as local bending deformation and macroscopic displacement of the seal membrane, rather than the overall microscopic molecular chain elongation and overall tensile deformation. And said inner portion 356, from said sealing lip 334 to said section 355, the change of shape is shown as the comprehensive effect of partial bending deformation and overall tensile deformation of the seal membrane. What it is quite clear is that said pleats enlarge hoop circumference, and reduce the cylinder hoop strain (stress) when a large diameter instrument is inserted, thereby reducing the hoop force and the frictional resistance.

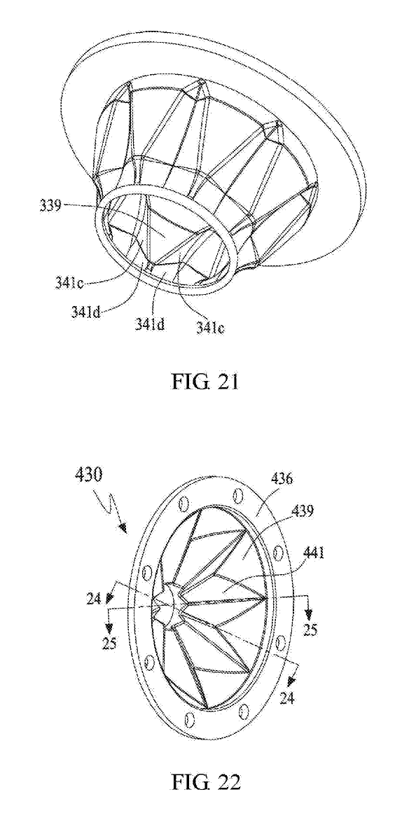

FIG. 20-21 shows a simulated deformation view of seal membrane 330 when a large diameter instrument is inserted. Said pleat wall 341 is divided into two portions, a pleat wall 341c and a cylinder 341d, wherein said cylinder 138d together forms the wrapped area around the outer surface of said inserted instrument. Studies have shown that, compared to the grooveless design, the wrapped area of the sealing body with the groove is small. Reducing the wrapped area can reduce the frictional resistance.

In an optional embodiment, thickened pleat peaks are adopted. Said thickened pleat peak, that is, the thickness of the wall at the pleat peak is greater than the thickness of the pleat wall. Said thickened pleat peak has the function of reinforcing ribs. In this embodiment, 8 thickened pleat peaks act as 8 reinforcing ribs, together to strengthen the axial tensile stiffness of the sealing wall 335. Since said pleats 340 enlarge the hoop circumference in the lip-adjacent area, the thickened pleat peaks enhance the axial tensile stiffness without significantly increasing the hoop tensile stiffness; that is, increasing the axial stiffness without increasing the hoop force, such that which can effectively reduce the stick-slip described in the background. In this embodiment, there are 8 thickened pleat peaks, while more or less which also can increase the axial tensile stiffness.

In summary, said pleats has the functions of enlarging hoop circumference, reducing the wrapped area, reducing the actual contact area of the two surfaces between the instrument and the seal membrane, increasing the axial tensile stiffness, etc., and therefore the frictional resistance and the stick-slip can be greatly reduced, and the probability of inversion is reduced.

As described in the background, when a 5 mm diameter instrument is inserted, it is considered only relying on the hoop force of the sealing lip to ensure sealing reliability. Therefore, it is not possible to reduce hoop strain (stress) by enlarging hoop circumference of the sealing lip when a large diameter instrument is inserted, however, the method of enlarge the hoop circumference can be used to reduce hoop strain (stress) in the lip-adjacent area. The strain in the lip-adjacent area is larger (high concentration stress area), and the closer to the sealing lip, the greater the strain (stress), and the closer to the sealing lip, the greater the strain (stress). Therefore, it is necessary to rapidly enlarging hoop circumference in the lip-adjacent area. However in the present embodiment, the larger the pleat angle θ, the rate of the hoop circumference in the lip-adjacent area enlarges. The pleat angle θ is the guide angle α, the guide angle β, and the number of pleats P, and conform to the following equation.

cos θ=cos α cos β cos(180/P)+sin α sin β

### Where:

cos=cosine function
sin=sin function
P=number of pleats
α=the angle between the pleat valley and the transverse plane
β=the angle between the pleat peak and the transverse plane
θ=the angle between the pleat peak and the pleat valley

Theoretically, the larger θ is, the better. That is it can quickly enlarge the hoop circumference in the lip-adjacent area, so the hoop force in pleats is fast minimized; while said hoop force is not the only factor that causes the frictional resistance to be large in the background. Rapidly reducing the hoop force in pleats, it is also necessary to comprehensively consider reducing the wrapping area and reducing the actual contact area of the two surfaces between the instrument and the seal membrane. By theoretical analysis and related research, it is shown that reducing the value of the guiding angle of the pleat wall in the lip-adjacent area (in this embodiment the guiding angle of the pleat wall is defined by the pleat valley guiding angle α and the pleat peak guiding angle β) is advantageous for reducing said wrapped area, but too small guiding angle will sacrifice the guiding performance of the seal membrane, therefore, when determining the value of the guiding angle, the smaller value should be taken as far as possible under the premise of satisfying the guiding performance.

According to the above equation, when the difference value (D-value) between α and β is the smallest, the equation on the right side of the equal sign takes the maximum value, that is, θ takes the minimum value. When the difference value between α and β is larger, the θ become larger. A smaller guide angle is advantageous to reduce the wrapped area. It is necessary to satisfy a large θ angle as well as satisfying a small introducing angle, so the smaller the angle α, the better. When the value of the angle α is determined, the value of β is selected according to the rate of increase of the circumferential circumference required for the design, that is, β is determined by the rate at which the height of the pleat wall increases. Optionally, in one embodiment, the geometric relationship of the pleats conforms to the following equation:

$$tan β - tan α \geq \sqrt{{( \frac{π {R}_{i}}{PR} )}^{2} + 2 ( cos ( 180 / P ) - 1 )}$$

### Where:

tan=cosine function
cos=cosine function
P=number of pleats
R=The distance from the pleat as the starting point for measurement to the central axis of the sealing lip
Ri=the largest radius designed for the surgical instrument through the seal membrane
β=the angle between the pleat peak and the transverse plane
α=the angle between the pleat valley and the transverse plane

It can be understood according to the above equation that a reasonable combination of R, α, β, P can make the region laterally outward from the measurement point, the change of which shape is mainly manifested by the local macroscopic displacement of the material, the produced strain (stress) is mainly manifested by local bending deformation, rather than the overall microscopic molecular chain elongation, thereby reducing the hoop force in a large extent. It can be understood according to the above equation that the larger the number of pleats P, the smaller the values of α, β angle can be selected, but in actual manufacturing, usually no more than 8 pleats, more pleats will make manufacturing very difficult or impossible to manufacture. Normally 2.5 mm≤R≤(Ri+R0)/2; Normally 2.0 mm≤R0≤2.2 mm: If the value of R is less than 2.5 mm, the transition area at the sealing lip will be too large; if the value of R is greater than (Ri+R0)/2, the effect of enlarging the hoop circumference in the lip-adjacent area and reducing the hoop force is not obvious. Optionally, the number of pleats P=8; the largest radius designed for the surgical instrument through the seal membrane Ri=6.45; the range of values is 3≤R≤4.

When R=3, α=0°, then β≥36.8°;
When R=3, α=20°, then β≥48.6°;
When R=3, α=25°, then β≥50.6°;
When R=3, α=30°, then β≥53°;
When R=4, α=0°, then β>31.5°;
When R=4, α=20°, then β≥44.4°;
When R=4, α=25°, then β≥47.20°;
When R=4, α=30°, then β≥50°.

Usually β should be less than or equal to 50°, and a larger β causes said wrapped area to increase. The above theoretical calculations have shown that with R (3≤R≤4) as the radius cylindrical surface intersects with pleats, when a large diameter instrument is inserted, pleats deformation in the inside of the cylinder is shown as the comprehensive effect of overall tensile deformation and local bending deformation; while the material of pleats in the outside of the cylinder is mainly manifested by local bending deformation and the overall displacement. When α>25°, to achieve the aforementioned effect, β should be greater than 50°, which will cause the wrapped area to be too large. Therefore, it is appropriate to be 0≤α≤25°.

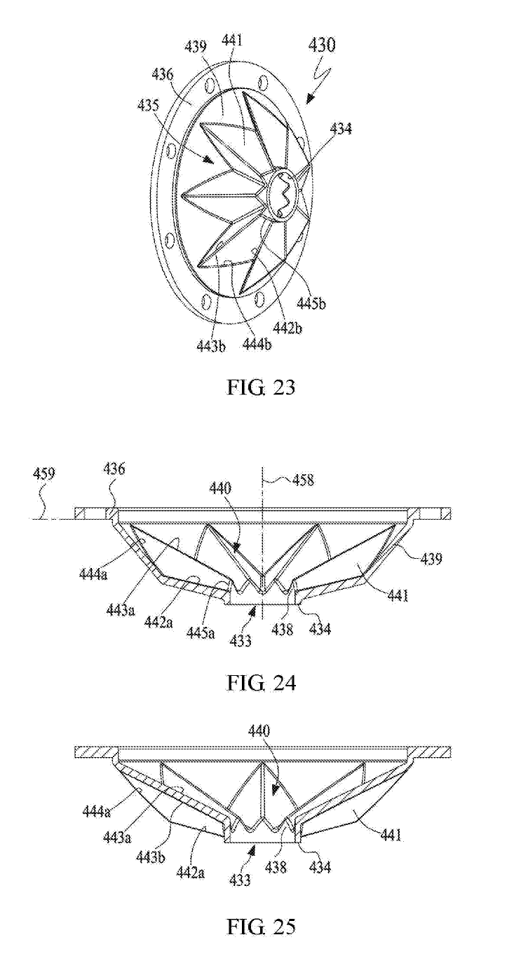

FIG. 23-25 show more detailed depiction the seal membrane 330 of the second embodiment of the invention. Said seal membrane 430 includes a proximal opening 432 (not shown), a distal end aperture 433, and the sealing wall extending from the distal end to the proximal end, said sealing wall including a proximal surface and a distal surface. Said aperture 433 formed by a sealing lip 434 for accommodating an inserted instrument and forming a gas-tight seal. Said the seal membrane 330 also including the flange 336; The sealing wall 335 has one end connected to the sealing lip 334 and the other end connected to the flange 336; the floating portion 337 (not shown) has one end connected to the flange 336 and the other end connected to said proximal end 332.

Defining the axis of said sealing lip 434 as the longitudinal axis 458, and a transverse plane 459 that is generally perpendicular to the longitudinal axis 458. Said sealing wall 435 includes a plurality of pleats 440. The pleats 440 and the sealing lip 434 are circumscribed and extend laterally away from the axis 458. Said pleats 440 include pleat valleys 442a, 442b; pleat peaks 443a, 443b; and a pleat wall 441. Sealing wall 435 includes 8 said linear pleats 440, in the present embodiment, although a more or less number of pleats can be employed. Said pleats 340 and the frustum wall 439 extend to be intersected and form an intersection line 444a, 444b; the frustum wall 339 and said flange 436 extend to be intersected.

When the pleats 340 extending laterally outward, the depth of said pleat wall 441 gradually increases along the longitudinal axis (in the lip-adjacent area the depth of pleats gradually increases), and then gradually decreases along the longitudinal axis (outside the lip-adjacent area the depth of said pleats gradually decreases). The height of the pleat wall can be measured along the wall surface between the pleat valley 442a (442b) and the pleat peak 443a (443b).

Said lip 434 has a cylindrical portion, which when intersected with the pleats 89 results in a line 445 a, 445 b; said line 445a (445b) defines a triangular region 338 pointing distally to the tip, corresponding to each peak 443a (443b).

In an optional embodiment, thickened pleat peaks are adopted. Said thickened pleat peak, that is, the thickness of the wall at the pleat peak is greater than the thickness of the pleat wall. Said thickened pleat peak has the function of reinforcing ribs. In this embodiment, 8 thickened pleat peaks act as 8 reinforcing ribs, together to strengthen the axial tensile stiffness of the sealing wall 435. Since said pleats 440 enlarge the hoop circumference in the lip-adjacent area, the thickened pleat peaks enhance the axial tensile stiffness without significantly increasing the hoop tensile stiffness; that is, increasing the axial stiffness without increasing the hoop force, such that which can effectively reduce the stick-slip described in the background. In this embodiment, there are 8 thickened pleat peaks, while more or less side walls also can increase the axial tensile stiffness. While the thickness of said frustum wall 439 is much smaller than the thickness of the pleat wall 441, which is mainly to reduce the deformation force outside the lip-adjacent area. When the seal membrane 440 is used in conjunction with the aforementioned protection device 160, the instrument is unlikely to contact said frustum wall 439, so a thinner thickness can be used without fear of damage to the seal membrane; said thicken pleat valley plays a role in increasing the axial tensile stiffness of the sealing wall 435, and therefore a thinner frustum wall 439 can be used to reduce the stress generated by the frustum wall 439 relative to the flange rotation and bending deformation when the sealing lip and its adjacent area are diastolic.

Likewise, said pleats has the functions of enlarging hoop circumference, reducing the wrapped area, reducing the actual contact area of the two surfaces between the instrument and the seal membrane, increasing the axial tensile stiffness, etc., and therefore the frictional resistance and the stick-slip can be greatly reduced, at the same time, the probability of inversion is reduced or the operational comfort of the seal membrane after inversion can be improved.

It will be readily apparent to those skilled in the art that a reasonable fillet transition can avoid stress concentration or easier deformation of certain areas. Since the diameter of the seal membrane is small, especially the diameter of the area near the sealing lip is smaller, such a small diameter and different chamfers that the appearance of the seal membrane looks different. In order to clearly show the geometric relationship of elements, the embodiment of the description in the invention is generally the graphics after removing fillet.

Many different embodiments and examples of the invention have been shown and described. Those ordinary skilled in the art will be able to make adaptations to the methods and apparatus by appropriate modifications without departing from the scope of the invention. The structure and the manner of fixing of the protector assembly disclosed in U.S. Pat. No. 7,788,861 are used in the example of the present invention. However, the structure and the manner of fixing of the protector assembly disclosed in U.S. Pat. No. 7,798,671 can be used, and in some applications, the protector assembly may not be included. It has been mentioned many times in the invention that the concave-furrow extends laterally outward from the sealing lip, and the so-called “extending laterally outward” should not be limited to a straight line. Said “extending laterally outward” can be a spiral, a line segment, a multi-section arc line and so on. In the invention, the positional relationship of the intersecting surfaces composed of said concave-furrow and the intersection line thereof are described with reference to specific embodiments, and the methods of increasing curved surfaces to form a multifaceted mosaic or using of the high-order curved surface to make the intersection line and the concave-furrow shape to look different from said embodiment. However, it can be considered not deviated from the scope of the invention, as long as it conforms to the general idea of the invention. Several modifications have been mentioned, to those skilled in the art, other modifications are also conceivable. Therefore, the scope of the invention should follow the additional claims, and at the same time, it should not be understood that it is limited by the specification of the structure, material or behavior illustrated and documented in the description and drawings.

I claim:

1. A trocar seal membrane for minimally invasive surgery, comprising:
a proximal opening, a distal aperture, and a sealing wall which extends from the distal aperture to the proximal opening, the distal aperture formed by a sealing lip for accommodating the inserted instrument and forming a seal gas-tight seal, the sealing lip comprising a longitudinal axis and a transverse plane substantially perpendicular to the longitudinal axis; and
the sealing wall comprises a plurality of pleats extending laterally from the sealing lip; each the pleat comprises a pleat peak, a pleat valley and a pleat wall extending there between; and
in the lip-adjacent area, the depth of the pleat wall gradually increases along the longitudinal axis, while outside the lip-adjacent area, the depth of which gradually decreases along the longitudinal axis; and the pleats geometric relationship conforms to the following equation:
$$tan β - tan α \geq \sqrt{{( \frac{π {R}_{i}}{PR} )}^{2} + 2 ( cos ( 180 / P ) - 1 )}$$
Where:
tan=tan function;
cos=cosine function;
P=number of pleats;
R=The distance from the pleat as the starting point for measurement to the central axis of the sealing lip;
Ri=the largest radius designed for the surgical instrument through the seal membrane;
β=the angle between the pleat peak and the transverse plane;
α=the angle between the pleat valley and the transverse plane.

2. The seal membrane of claim 1, comprising eight pleats.

3. The seal membrane of claim 1, wherein the sealing lip is circular and its radius R0, and 2.0≤R0≤2.2 Millimeters.

4. The seal membrane of claim 3, wherein 2.5 mm≤R≤(Ri+R0)/2.

5. The seal membrane of claim 1, wherein 0°≤α≤25°.

6. The seal membrane of claim 1, wherein 3 mm≤R≤4 mm.

7. The seal membrane of claim 1, wherein the thickness of the wall at the pleat peaks is greater than the thickness of the pleat wall, the pleat peak function as reinforcing ribs to enhance the axial tensile stiffness without significantly increasing the hoop tensile stiffness.

8. The seal membrane of claim 1, wherein the thickness of the wall at the pleat valleys is greater than the thickness of the pleat wall, the pleat valleys function as reinforcing ribs to enhance the axial tensile stiffness without significantly increasing the hoop tensile stiffness.

9. The seal membrane of claim 8, the sealing wall comprising a flange and a conical side wall extending from the flange; the conical side wall and the pleats are intersected; the thickness of the conical side wall is less than the thickness of the pleat wall which benefits of reducing the force generated by the conical side wall relative to the flange rotation and bending deformation.

10. The seal membrane of claim 1, wherein the seal membrane also includes a flange and an outer floating portion extending from the flange to the proximal opening.

11. A trocar seal assembly, wherein the seal membrane comprises the seal membranes as defined in claim 10, and including a lower retainer ring, a upper retainer ring, a protection device, an upper body and an upper cover, the seal membrane and the protect device are sandwiched between the upper retainer ring and the lower retainer ring, the seal membrane also includes a flange and an outer floating portion extending from the flange to the proximal opening, and the proximal opening are sandwiched between the upper body and the upper cover.
